(19) United States
(12) Patent Application Publication
(10) Pub. No.: US 2005/0070974 A1
(43) Pub. Date: Mar. 31, 2005

(54) Obesity and eating disorder stimulation treatment with neural block

(76) Inventors: Mark B. Knudson, Shoreview, MN (US); Richard R. Wilson, Arden Hills, MN (US); Katherine S. Tweden, Mahtomedi, MN (US); Timothy R. Conrad, Eden Prairie, MN (US)

Correspondence Address: Merchant & Gould P.C., P.O. Box 2903, Minneapolis, MN, 55402-0903, US

(21) Appl. No.: 10/756,176

(22) Filed: Jan. 12, 2004

Related U.S. Application Data
(63) Continuation-in-part of application No. 10/674,330, filed on Sep. 29, 2003.
(63) Continuation-in-part of application No. 10/675,818, filed on Sep. 29, 2003.
(63) Continuation-in-part of application No. 10/674,324, filed on Sep. 29, 2003.
(63) Continuation-in-part of application No. 10/752,944, filed on Jan. 6, 2004.
(63) Continuation-in-part of application No. 10/752,940, filed on Jan. 6, 2004.

(51) Int. Cl. A61N 1/18

(52) U.S. Cl. 607/58

(57) ABSTRACT

A method and apparatus for treating patients suffering from obesity or eating disorders by applying a predetermined stimulating signal to the patient's vagus nerve appropriate to alleviate the condition and by applying a neural conduction block to the vagus nerve at a blocking site with the neural conduction block selected to at least partially block nerve impulses on the vagus nerve at the blocking site.

### I. CROSS-REFERENCE TO RELATED APPLICATIONS

This application is a continuation-in-part of the following U.S. patent applications, each filed Sep. 29, 2003: Ser. No. 10/674,330 titled “Nerve Conduction Block Treatment”; Ser. No. 10/675,818 titled “Enteric Rhythm Management” and Ser. No. 10/674,324 titled “Nerve Stimulation And Conduction Block Therapy”. The present application is also a continuation-in-part of U.S. Ser. No. [not yet assigned], attorney docket number 14283.1US14 titled “Electrode Band Apparatus and Method” and U.S. Ser. No. [not yet assigned], attorney docket number 14283.1 USI5 titled “Intraluminal Electrode Apparatus and Method”, each filed Jan. 6, 2004 in the names of the same inventors as in the present application.

### II. BACKGROUND OF THE INVENTION

1. Field of the Invention

This invention pertains to electrical stimulation treatments for treating obesity and/or eating disorders. More particularly, this invention pertains to an improvement to prior art treatments by adding neural conduction blocks to such treatments.

2. Description of the Prior Art

A prior art method and apparatus for treating an eating disorder by nerve stimulation (e.g., electrical stimulation applied to the vagus nerve) are disclosed in U.S. Pat. No. 5,188,104 to Wernicke et al. dated Feb. 23, 1993 (the “‘104 patent’”) and U.S. Pat. No. 5,263,480 to Wernicke et al. dated November 23, 1993 (the “‘480 patent’”). A prior art method and apparatus for treating obesity by nerve stimulation (e.g., electrical stimulation applied to the vagus nerve) are disclosed in U.S. Pat. No. 6,609,025 to Barret et al. dated Aug. 19, 2003 (the “‘025 patent’”) and U.S. Pat. No. 6,587,719 to Barret et al. dated November 23, 1993 (the “‘719 patent’”).

Applicants believe a best approach to obesity treatment involves applying a neural block to the vagus instead of stimulating the vagus as described in the '025 and '719 patents. Applicants' blocking treatment is the subject of patent applications referenced in the first paragraph of this application (“Cross-Reference to Related Applications”). Nevertheless, others have reported some success with stimulation treatments for obesity and sleep disorder as described in the afore-mentioned prior art patents. The present application is directed to improvements of the stimulation techniques described in those patents to avoid adverse effects of nerve stimulation on other organs.

A problem associated with nerve stimulation is the creation of undesired side effects. For example, stimulation of the vagus nerve can create undesired cardiac or voice responses. Stimulation near a diaphragm can have cardiopulmonary effect as well as undesired gastrointestinal effects or pancreobiliary effects. Another potential problem associated with nerve stimulation is that antidromic inhibitory responses may interfere with the effectiveness of the procedure.

U.S. Pat. No. 5,205,285 to Baker, Jr. dated Apr. 27, 1993 describes voice suppression of vagal stimulation as an attempt to address the issue of unwanted side effects. The '285 patent states that in at least some patients receiving vagal stimulation treatment for epileptic seizures, there is a noticeable modulation of speech during actual application of the stimulation. According to the teachings of U.S. Pat. No. 5,205,285 (incorporated herein by reference), the vagal stimulation for seizure treatment is de-activated during periods of speech.

Unwanted side effects can also be addressed by lowering the energy levels of stimulation or reducing the duration over which stimulation therapy is applied. Both of these reduce the efficacy of treatment.

Another technique for addressing the side effects is to permit a patient to control when a stimulation is applied. A patient activation of stimulation therapy is described in U.S. Pat. No. 5,304,206 to Baker Jr., et al. dated Apr. 19, 1994. Again, by the time a patient senses a need for therapy, the ability to effectively intervene may be compromised. Furthermore, patient control is unreliable.

An object of the present invention is to provide a neural conduction block to the vagas in combination with stimulation to block signals at the blocking site. The present invention describes a blocking of a nerve (such as the vagal nerve) to avoid antidromic influences during stimulation or to block stimulation signals which might otherwise result in adverse side effects. Cryogenic nerve blocking of the vagus is described in Dapoigny et al., “Vagal influence on colonic motor activity in conscious nonhuman primates”, Am. J. Physiol., 262: G231-G236 (1992). Electrically induced-nerve blocking is described in Van Den Honert, et al., “Generation of Unidirectionally Propagated Action Potentials in a Peripheral Nerve by Brief Stimuli”, Science, Vol. 206, pp. 1311-1312. An electrical nerve block is described in Solomonow, et al., “Control of Muscle Contractile Force through Indirect High-Frequency Stimulation”, Am. J. of Physical Medicine, Vol. 62, No. 2, pp. 71-82 (1983) and Petrofsky, et al., “Impact of Recruitment Order on Electrode Design for Neural Prosthetics of Skeletal Muscle”, Am. J. of Physical Medicine, Vol. 60, No. 5, pp. 243-253 (1981). A neural prosthesis with an electrical nerve block is also described in U.S. patent application Publication No. US 2002/0055779 A1 to Andrews published May 9, 2002. A cryogenic vagal block and resulting effect on gastric emptying are described in Paterson CA, et al., “Determinants of Occurrence and Volume of Transpyloric Flow During Gastric Emptying of Liquids in Dogs: Importance of Vagal Input”, Dig Dis Sci, (2000);45:1509-1516.

### III. SUMMARY OF THE INVENTION

According to a preferred embodiment of the present invention, a method and apparatus are disclosed for treating obesity or eating disorders by applying a predetermined stimulating signal to the patient's vagus nerve appropriate to alleviate the condition and by applying a neural conduction block to the vagus nerve at a blocking site with the neural conduction block selected to at least partially block nerve impulses on the vagus nerve at the blocking site.

### IV. BRIEF DESCRIPTION OF THE DRAWINGS

FIG. 1 is a simplified block diagram of an implantable neurostimulator electronics package (stimulus generator) for use (with appropriate parameter settings and ranges) in treating eating disorders according to the teachings of U.S. Pat. Nos. 5,188,104 and 5,263,480;

FIG. 2 is a simplified fragmentary illustration of the stimulus generator and lead/electrode system of the neurostimulator implanted in the patient's body as taught in the '104 and '480 patents;

FIG. 3 is a more detailed view of a portion of the preferred embodiment of the stimulus generator and associated lead/electrode system of the neurostimulator illustrating certain exemplary details of the generator and of the placement of the detection and stimulation portions of the lead/electrode system relative to the patient's vagus nerve and stomach as taught in the '104 and '480 patents;

FIG. 4 is a detailed fragmentary illustration of the implanted nerve electrode for modulating vagal activity as taught in the '104 and '480 patents;

FIG. 5 is an illustrative idealized electrical output signal waveform of the stimulus generator useful for clarifying relevant parameters of the signal as taught in the '104 and '480 patents;

FIG. 6 is a view of FIG. 2 modified according to the teachings of the present invention;

FIG. 7 is a simplified fragmentary illustration of the stimulus generator and lead/electrode system of the neuro stimulator implanted in the patient's body as taught in U.S. Pat. No. 6,587,719;

FIG. 8 is a view of FIG. 7 modified according to the teachings of the present invention;

FIG. 9 is a simplified partial front view of a patient (in phantom) having an implanted neurostimulator for generating the desired signal stimuli which are applied directly and bilaterally at sub-diaphragmatic location to the right and left branches of the patient's vagus via an implanted lead/nerve electrode system electrically connected to the neurostimulator as taught in U.S. Pat. No. 6,609,025;

FIG. 10 is a simplified partial front view of a patient similar to that of FIG. 9, but in which a pair of implanted neurostimulators is used for generating the desired signal stimuli as taught in the '025 patent;

FIG. 11 is a simplified partial front view of a patient in which an implanted neurostimulator and associated electrode is used for unilateral stimulation of only one branch of the vagus nerve as_taught in the '025 patent;

FIG. 12 is a simplified partial front view of a patient in which the signal stimuli are applied at a portion of the nervous system remote from the vagus nerve such as at or near the stomach wall, for indirect stimulation of the vagus nerve as taught in the '025 patent;

FIG. 13 is a simplified partial front view of a patient in which the signal stimuli are applied remotely from electrical stimulating device placed by an endoscope from an area composing the GI tract as taught in the '025 patent;

FIG. 14 is the view of FIG. 9 modified according to the teachings of the present invention;

FIG. 15 is the view of FIG. 10 modified according to the teachings of the present invention;

FIG. 16 is the view of FIG. 11 modified according to the teachings of the present invention;

FIG. 17 is the view of FIG. 12 modified according to the teachings of the present invention; and

FIG. 18 is the view of FIG. 13 modified according to the teachings of the present invention.

### V. DESCRIPTION OF THE INVENTION

Referring now to the several drawing figures in which identical elements are numbered identically throughout, a description of a preferred embodiment of the present invention will now be provided. For ease of understanding, a description of the prior art as appears in prior art patents will first be provided following by a description of the present invention.

The disclosures of the following patents are incorporated herein by reference: U.S. Pat. No. 5,188,104 to Wernicke et al. dated Feb. 23, 1993 (the “‘104 patent’”); U.S. Pat. No. 5,263,480 to Wemicke et al. dated Nov. 23, 1993 (the “‘480 patent’”); U.S. Pat. No. 6,609,025 to Barret et al. dated Aug. 19, 2003 (the “‘025 patent’”) and U.S. Pat. No. 6,587,719 to Barret et al. dated Nov. 23, 1993 (the “‘719 patent’”). In the sections of this application pertaining to teachings of the prior art, the specification from prior art patents is substantially reproduced for ease of understanding the embodiment of the present invention. For the purpose of the present application, Applicants accept the accuracy of information in those patents without independent verification.

A. Teachings of Prior Art

For ease of illustrating the present invention in a preferred embodiment for improving a prior art system for treating obesity and eating disorders in a first prior art embodiment, a recitation of the invention of U.S. Pat. No. 5,188,104 to Wemicke et al. dated February 23, 1993 (the “‘104 patent’”) and U.S. Pat. No. 5,263,480 to Wemicke et al. dated Nov. 23, 1993 (the “‘480 patent’”) is first provided.

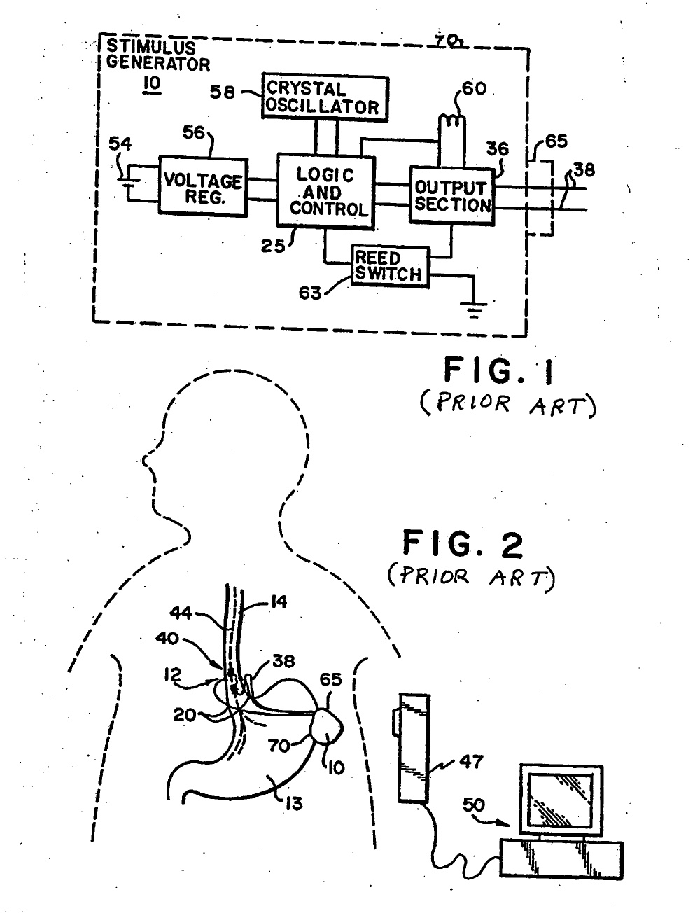

Referring now to FIGS. 1-5, a block diagram of the basic components of the general electronics package of an implantable neurostimulator and their interrelationship is illustrated in FIG. 1, and details of location of the implanted device and of portions of the preferred embodiment of the electronics package and lead/electrode system are shown in FIGS. 2, 3 and 4. A generally suitable form of neurostimulator for use in the apparatus of the present invention is disclosed in copending U.S. Pat. No. 5,154,172 to Terry et al., dated Oct. 13, 1992 (referred to herein as the “'172 patent”), assigned to the same assignee as the instant application. The specification of the '172 patent is incorporated herein in its entirety by reference, but for the sake of convenience to the reader, certain portions of it are summarized in this application.

The neurostimulator utilizes a conventional microprocessor and other standard electrical and electronic components, and communicates with an external programmer and/or monitor by asynchronous serial communication for controlling or indicating states of the device. Passwords, handshakes and parity checks are employed for data integrity. The neurostimulator also includes means for conserving energy, which is important in any battery operated device and especially so where the device is implanted for medical treatment of a disorder, and means for providing various safety functions such as preventing accidental reset of the device.

A preferred embodiment of the neurostimulator according to the '104 patent has certain material differences from the type described in detail in the '172 patent, as will be described below. An electronics package in the form of stimulus generator 10 is implanted in the patient's body, preferably in a pocket formed by the implanting surgeon just below the skin in the abdomen as shown in FIG. 2. In conjunction with its microprocessor-based logic and control circuitry, stimulus generator 10 includes detection circuitry for automatically initiating the stimulating signal generation, and output circuitry for patterning the stimulating signal to modulate vagal activity in a manner designed to treat the compulsive eating disorder of interest.

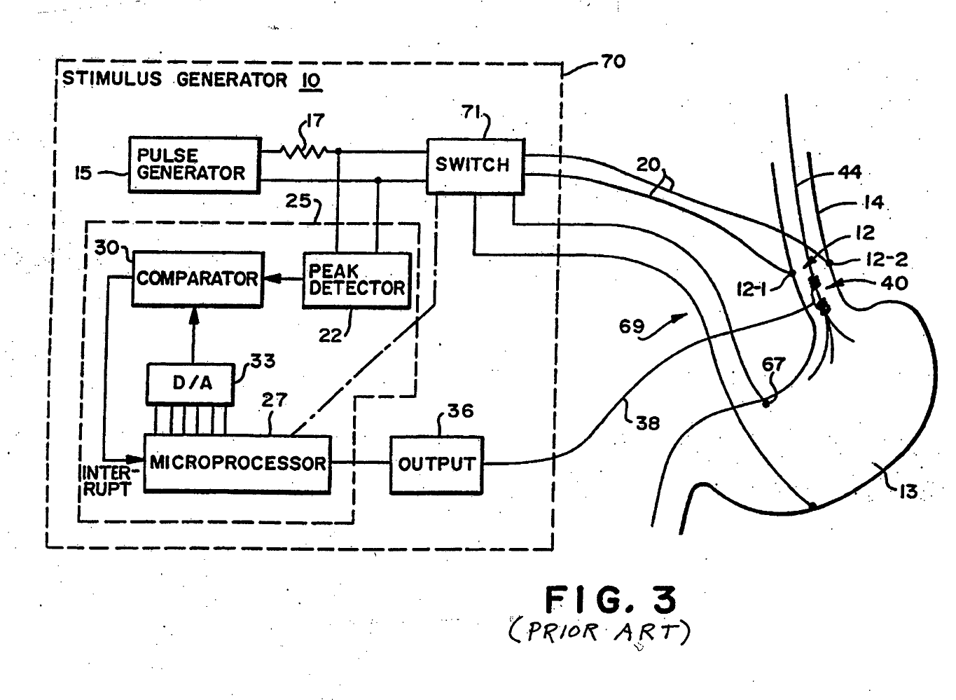

The detection circuitry includes a set of implantable electrodes 12 (FIG. 2) which are coupled to the stimulus generator by a suitable electrical lead or leads 20 of known type for use in and biocompatible with implantation in the body. Electrodes 12 themselves are secured to opposite sides of the patient's esophagus 14, preferably at a site just above the stomach 13 as shown in FIGS. 2 and 3. Alternatively, they may be located in the patient's neck. Electrode set 12 may be bipolar or quadripolar, to sense the impedance between one pair or two pairs of electrodes positioned at the opposite sides of esophagus 14 generally in the same plane and normal to the length of the esophageal tube. The electrodes themselves may be composed of activated iridium, rhodium, platinum or other suitable material.

Because polarization potentials on the electrodes tend to distort the measurements, it is desirable to employ quadripolar electrodes in which one pair is used for signal generation and the other pair is used for signal sensing. Preferably, however, each of the detecting electrodes is coated with a thin layer of iridium oxide to substantially enhance its sensitivity to electrical signals, reduce polarization potentials, and rapidly dissipate the polarization potentials. If the iridium oxide coating is used, a bipolar electrode set 12-1, 12-2 is preferred (FIG. 3). Each of the electrodes may be provided with a biocompatible fabric “collar” or band about the electrode periphery to allow it to be readily sutured in place in the esophageal locations.

With reference to FIG. 3, stimulus generator 10 includes a pulse generator 15 (preferably, but an AC signal generator may alternatively be used). With a four electrode system, the pulse generator is connected via a high impedance 17 and the leads 20 to excitation electrodes, and a peak detector 22 is connected via leads 20 to sensing electrodes, among electrode set 12. The peak detector includes a low pass filter for smoothing the detected peaks over a predetermined period of time—ten seconds, for example—although the averaging period may be more or less than that as will be apparent from the description of operation set out below. The peak detector and averaging circuit are part of the logic and control section 25 of the stimulus generator electronics package (FIGS. 1 and 3). Section 25 also includes a microprocessor 27, a comparator 30 and a digital-to-analog (D/A) converter 33 (FIG. 3).

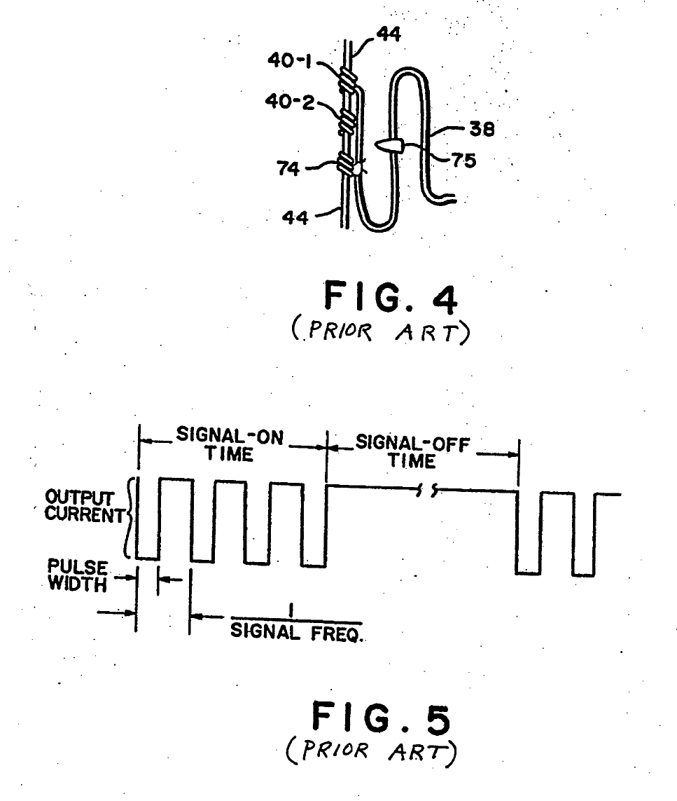

The output of the peak detector 22 is applied as one input to comparator 30, which also receives an input in the form of analog data from the D/A converter 33. Microprocessor 27, which is programmable, supplies digital inputs to the D/A converter and receives an input from comparator 30. The microprocessor supplies a control input, as an output of logic and control section 25, to an output signal generation section 36. The latter is coupled to bipolar stimulation electrode set 40 via a biocompatible electrical lead or leads 38. Stimulation electrode set 40 is secured to the patient's vagus nerve 44 (FIG. 3, and in greater detail in FIG. 4).

Components of the system for use (by the attending physician) external to the patient's body including a programming wand 47 which, among other things, communicates parameter changes to stimulus generator 10, and a computer 50 and associated software for adjustment of parameters and control of communication between the implanted electronics, the programming wand and the computer (FIG. 2).

As shown in more simplified block diagrammatic form in FIG. 1, stimulus generator 10 also includes a battery (or set of batteries) 54, which may be of any reliable long-lasting type conventionally employed for powering implantable medical electronic devices (such as batteries employed in implantable cardiac pacemakers or defibrillators). In the preferred embodiment of the stimulus generator of the '104 patent, battery 54 is a single lithium thionyl chloride cell with its output terminals connected to the input side of voltage regulator 56. The regulator smoothes the battery output to produce a clean, steady output voltage, and provides enhancement thereof such as voltage multiplication or division if necessary for a specific application.

The regulator 56 supplies power to logic and control section 25, which controls the programmable functions of the device. Among these programmable functions are output current, output signal frequency, output signal pulse width, output signal on-time, output signal off-time, daily treatment time for periodic modulation of vagal activity), and output signal-start delay time. Such programmability allows the output signal to be selectively crafted for application to the stimulating electrode set 40 (FIG. 2) to obtain the desired modulation of vagal activity for treatment and control of the eating disorder of interest with the particular patient. Logic and control section 25 may also be implemented to control programmable functions of the pulse generator 15 (FIG. 3). Timing signals to section 25 and to pulse generator 15 are provided by a crystal oscillator 58.

Built-in antenna 60 enables communication between the implanted stimulus generator and the external electronics (including both programming and monitoring devices) to permit the device to receive programming signals for parameter changes, and to transmit telemetry information, from and to programming wand 47. Once the system is programmed, it operates continuously at the programmed settings until they are reprogrammed by means of computer 50 and programming wand.

The logic and control section controls an output circuit or section 36 of the stimulus generator, which functions to generate the programmed signal levels appropriate to the condition (eating disorder) being treated. Output section 36 and the programmed output signal thereof is coupled (directly, capacitively, or inductively) to an electrical connector 65 on the housing 70 of the generator and to the lead assembly 38 and the stimulating electrodes 40 connected thereto (FIG. 2). In this way, the programmed output signal of stimulus generator 10 is applied to the nerve electrode set implanted on the patient's vagus nerve 44, to modulate the vagal activity in a desired manner to alleviate the disorder.

A reed switch 63 (FIG. 1) permits alternative or additional manual activation of the implanted electronics package by the patient, by placement of an external magnet (not shown) in proximity to the implanted device. Other forms of manual activating means may be employed instead, such as a microphone for detecting taps by the patient on the skin directly over the stimulus generator, or a patient-triggered external RF signal generator.

The entire stimulus generator 10 is housed in a hermetically sealed, biologically compatible (biocompatible) titanium case indicated by the dotted line 70 (FIG. 1). Further details of suitable structure and operation of the neurostimulator, beyond those by which the device is adapted to treat the selected eating disorder as described herein, are available in the '985 application, to which the reader is referred.

In operation of the stimulus generator 10 to control and treat compulsive overeating (including binge eating), the pulsed signal from pulse generator 15 is applied to excitation/sensing electrodes 12-1, 12-2 via high impedance 17 and lead 20 (FIG. 3). The amplitude of the signal on these electrodes is a function of the impedance between them, which varies according to whether the esophagus 14 is empty or has food passing through it (and therefore, between the electrodes). The peak signal amplitude on electrodes 12 is detected and averaged by peak detector 22 over a predetermined interval of time. This may be calibrated to differentiate between different types of swallowing, such as of solids versus liquids and/or short swallows versus long swallows. The period of time in question may be selected according to the individual patient's eating habits.

The number and length of swallows occurring within the predetermined interval is detected by the esophageal electrode/detection system, and the sum of the swallows is calculated by processing to estimate the quantity of food consumed by the patient. The peak detector and averaging circuit 22 smooth each swallow derived from the parameters of the electrical signal on the lead/electrode system, and the comparator 30 detects the presence and length of the swallows from that information and information supplied by microprocessor 27 via D/A converter 33. The microprocessor, in turn, sums the number of swallows in the predetermined interval and compares that number to a programmed threshold value, representative of a known quantity of consumption. When the summed number reaches or exceeds the programmed threshold value, the microprocessor initiates the stimulation signal for application to the nerve electrode set 40 implanted on the vagus nerve, by selective activation of the output signal generator 36.

Alternatively, or in addition to the sensing electrodes 12 on the patient's esophagus, a set of bipolar electrodes 67 secured to the outer wall of the patient's stomach 13 (one at each of opposite sides as depicted in FIG. 3) may be utilized for purposes of measuring the amount of food in the stomach. The stomach electrodes would be connected via lead(s) 69, and selectively through a double pole switch 71 controlled by the microprocessor in the stimulus generator, to the pulse generator and the peak detector. The stomach impedance sensing electrode system and its operation is similar to that described above for the esophagus impedance sensing electrode system. However, the body generates digestive fluids in response to the presence of food, and hence, the impedance changes in the stomach are of a more complex nature than those observed at the esophageal electrodes. A combination of these sensing electrode systems, one on the esophagus and one on the stomach, may provide better data to the microprocessor than either alone, to more accurately determine the food intake and amount of food in the stomach. Such an arrangement, however, requires the use of the additional lead and electrode set (69, 67) for the stomach impedance sensing, and of the double pole switch 71 controlled by microprocessor 27 in the stimulus generator, and is not part of the preferred embodiment.

The detection system may be and preferably is calibrated by telemetry (via programming wand 47) to the implanted neurostimulator for each individual patient and the specific nature of the eating disorder. The results are then programmed into the microprocessor for the appropriate treatment.

As discussed above, the stimulus generator may also be activated manually by the patient by any of various means by appropriate implementation of the device. These techniques include the patient's use of an external magnet, or of an external RF signal generator, or tapping on the surface overlying the stimulus generator, to activate the neurostimulator and thereby cause the application of the desired modulating signal to the stimulating electrodes. Upon experiencing the compulsive craving, the obese or bulimic patient can simply voluntarily activate the stimulus generator. If the patient fails to act, the automatic detection of the overeating and consequent application of the necessary therapy will take place through modulation of vagal activity to produce the sensation of satiety.

Another form of treatment of compulsive overeating may be implemented by programming the stimulus generator to periodically deliver the vagal activity modulation productive of satiety at programmed intervals between prescribed normal mealtimes. This will tend to reduce excessive snacking between meals, which may otherwise be of insufficient quantity within a preset time interval to trigger automatic delivery of the therapy. It will be noted that the various techniques employed according to the methods and apparatus of the present invention are designed to treat the symptoms of the disorder rather than to target the root cause of the compulsive behavior. In essence, the patient is “tricked” into believing that the symptom of the eating disorder is not present, by the sensation of reducing or enhancing the appetite depending on the nature of the eating disorder being treated and the consequent programming of the stimulating signal parameters. Nevertheless, these types of treatment can be very beneficial, particularly in extreme cases.

Features may be incorporated into the neurostimulator for purposes of the safety and comfort of the patient. The patient's comfort would be enhanced by ramping the stimulus up during the first two seconds of stimulation. The device may also have a clamping circuit to limit the maximum voltage (14 volts for example) deliverable to the vagus nerve, to prevent nerve damage. An additional safety function may be provided by implementing the device to cease stimulation in response to manual deactivation through techniques and means similar to those described above for manual activation. In this way, the patient may interrupt the stimulation if for any reason it suddenly becomes intolerable.

The stimulating nerve electrode set or assembly 40 is shown in greater detail in FIG. 4. The electrode set is conductively connected to the distal end of a pair of insulated electrically conductive electrode leads 38 which are attached at the proximal end to the connector 65 (and thereby, to the output signal generating circuit 36) of the electronics package. Electrode set 40 comprises bipolar stimulating electrodes 40-1 and 40-2, preferably of the type described in U.S. Pat. No. 4,573,481 issued Mar. 4, 1986 to Bullara. The electrode assembly is surgically implanted around the vagus nerve 44 in the patient's abdomen just above the stomach. The two electrodes 40-1 and 40-2 are wrapped about the vagus nerve, and the assembly is secured to the nerve by a spiral anchoring tether 74 preferably as shown in U.S. Pat. No. 4,979,511 issued Dec. 25, 1990 to Reese S. Terry, Jr. Lead(s) 38 is secured, while retaining the ability to flex with movement of the chest and abdomen, by a suture connection 75 to nearby tissue.

The open helical design of electrode assembly 40 (described in detail in the above-cited Bullara patent), which is self-sizing and flexible, minimizes mechanical trauma to the nerve and allows body fluid interchange with the nerve. The electrode assembly conforms to the shape of the nerve, providing a low stimulation threshold by allowing a larger stimulation contact area. Structurally, the electrode assembly comprises two ribbons of platinum constituting the electrodes which are individually bonded to the inside surface of each of the first two spiral loops 40-1 and 40-2 of a three-loop helical assembly, and the two lead wires are respectively welded to the conductive ribbon electrodes. The remainder of each loop is composed of silicone rubber, and the third loop 74 acts merely as the tether for the electrode assembly. The inner diameter of the helical bipolar electrode assembly 40 may typically be approximately two millimeters (mm) and an individual spiral is about seven mm long (measured along the axis of the nerve).

The stimulus generator may be programmed with programming wand 47 and a personal computer 50 using suitable programming software developed according to the programming needs and signal parameters which have been described herein. The intention, of course, is to permit noninvasive communication with the electronics package after the latter is implanted, for both monitoring and programming functions. Beyond the essential functions, the programming software should be structured to provide straightforward, menu-driven operation, HELP functions, prompts, and messages to facilitate simple and rapid programming while keeping the user fully informed of everything occurring at each step of a sequence. Programming capabilities should include capability to modify the electronics package's adjustable parameters, to test device diagnostics, and to store and retrieve telemetered data. It is desirable that when the implanted unit is interrogated, the present state of the adjustable parameters is displayed on the PC monitor so that the programmer may then conveniently change any or all of those parameters at the same time; and, if a particular parameter is selected for change, all permissible values for that parameter are displayed so that the programmer may select an appropriate desired value for entry into the neurostimulator.

Other desirable features of appropriate software and related electronics would include the capability to store and retrieve historical data, including patient code, device serial number, number of hours of battery operation, number of hours of stimulation output, and number of magnetic activations (indicating patient intercession) for display on a screen with information showing date and time of the last one or more activations.

Diagnostics testing should be implemented to verify proper operation of the device, and to indicate the existence of problems such as with communication, the battery, or the lead/electrode impedance. A low battery reading, for example, would be indicative of imminent end of life of the battery and need for implantation of a new device. However, battery life should considerably exceed that of other implantable medical devices, such as cardiac pacemakers, because of the relatively less frequent need for activation of the neurostimulator of the present invention. In any event, the nerve electrodes are capable of indefinite use absent indication of a problem with them observed on the diagnostics testing.

FIG. 5 illustrates the general nature, in idealized representation, of the output signal waveform delivered by output section 36 of the neurostimulator to electrode assembly 40. This illustration is presented principally for the sake of clarifying terminology, including the parameters of output signal on-time, output signal off-time, output signal frequency, output signal pulse width, and output signal current. Such parameters are discussed below in terms of ranges of values and typical values of the output signal which may be programmed into the device for treatment of various eating disorders.

For the obese patient, the stimulation strategy programmed into the neurostimulator is to provide modulation through the medium of the stimulating signal which is appropriate to increase vagal activity before and during meal periods. In the preferred embodiment and method of the invention, the stimulus generator output signal may be patient activated, but means are provided to detect eating, as the distension of and presence of food in the esophagus during swallowing and to integrate the number of swallows over time to detect the amount of food consumed, and when that amount exceeds a predetermined quantity in the selected time interval, to trigger automatic activation of the output signals from the electronics package.

The preferred range of stimulation parameters of the output signal for treatment and control of eating disorders, and the nominal value of each parameter programmed into the device by the attending physician are set forth in the following table.

| | Range | Typical |
|---|---|---|
| Pulse Width | 0.05-1.5 ms | 0.5 ms |
| Output Current | 0.1-5.0 mA | 1.5 mA |
| Frequency | 5-150 Hz | 25 Hz |
| On Time | 300-10,000 sec | 300 sec |
| Off Time | 300-30,000 sec | 1000 sec |
| Frequency Sweep? | 10-50 Hz | Yes (optional) |
| Random frequency? | 10-50 Hz | Yes (optional) |

The device may utilize circadian or other programming as well, so that activation occurs automatically at normal mealtimes for this patient. This may be in addition to the provision for the manual, periodic between meal, and sensing-triggered activation as described above herein. The treatment induces appetite suppression in obese patients and contributes ultimately to weight reduction, by controlling (producing the sensation of) satiety in the patient.

For bulimia patients, the device is programmed in the same manner as above, so that when triggered, vagal activity is increased and the patient's appetite is suppressed by a feeling of fullness. Manual activation by the patient is desirable, but because the psychological pattern is difficult to control, the use of circadian programming and detection of overeating by measuring quantity of food consumed during a given interval serves as an important backup in the therapeutic modality. It is also desirable to decrease vagal activity at other times to provide some smoothing out of eating.

In the treatment of anorexia nervosa patients, the programming is set to modulate vagal activity to considerably enhance the patient's appetite and the urge to eat, or at least to suppress satiety. Here also, the device may be manually activated, automatically activated upon detection of the condition of “emptiness” of the stomach or of exceeding a certain period since the last swallowing of food, or activated according to the patient's circadian cycle to induce hunger (or suppress satiety) at normal mealtimes, or by a combination of such activations.

As noted earlier herein, inhibition or blocking of signals on the vagus nerve is a potential strategy for treating or controlling an eating disorder such as anorexia nervosa. However, vagal stimulation generates a signal on the nerve, and the stomach signals are carried by the small C fibers which become refractory if stimulated at high frequency (for example, 40 Hz or higher) for more than a period of 30 to 60 seconds. Therefore, a strategy for inhibiting or blocking this C-fiber information is to stimulate the high frequencies with on-time of, say, 300 seconds and off-time of about 20 seconds. This sequence would be repeated for the interval of time that control (blocking of the C-fiber information) is desired to be exercised.

Alternatively, because C fibers become refractory if stimulated for a sufficiently long period, another strategy would be to continuously stimulate the C fibers to render them refractory and thereby block the nerve signals from getting through. The signals of interest are believed to be conducted principally if not solely on the C fibers. These fibers are slow to conduct compared to the A and B fibers, but the slower response is acceptable here. An important aspect is the programming of stimulation parameters which block vagal activity despite the speed of conduction of the nerve.

Although a totally implantable device is preferred by the '104 patent, the electronic energization package may, if desired, be primarily external to the body. Stimulation can be achieved with RF power device providing the necessary energy level. The implanted components could be limited to the lead/electrode assembly, a coil and a DC rectifier. With such an arrangement, pulses programmed with the desired parameters are transmitted through the skin with an RF carrier, and the signal is thereafter rectified to regenerate a pulsed signal for application as the stimulus to the vagus nerve to modulate vagal activity. This would virtually eliminate the need for battery changes.

However, the external transmitter must be carried on the person of the patient, which is inconvenient. Also, detection is more difficult with a simple rectification system, and greater power is required for activation than if the system were totally implanted. In any event, a totally implanted system is expected to exhibit a relatively long service lifetime, amounting potentially to several years, because of the relatively small power requirements for most eating disorder treatment applications. Also, as noted earlier herein, it is possible, although considerably less desirable, to employ an external stimulus generator with leads extending percutaneously to the implanted nerve electrode set. The major problem encountered with the latter technique is the potential for infection. Its advantage is that the patient can undergo a relatively simple procedure to allow short term tests to determine whether the eating disorder of this particular patient is amenable to successful treatment. If it is, a more permanent implant may be provided.

B. Improvement of the Present Invention

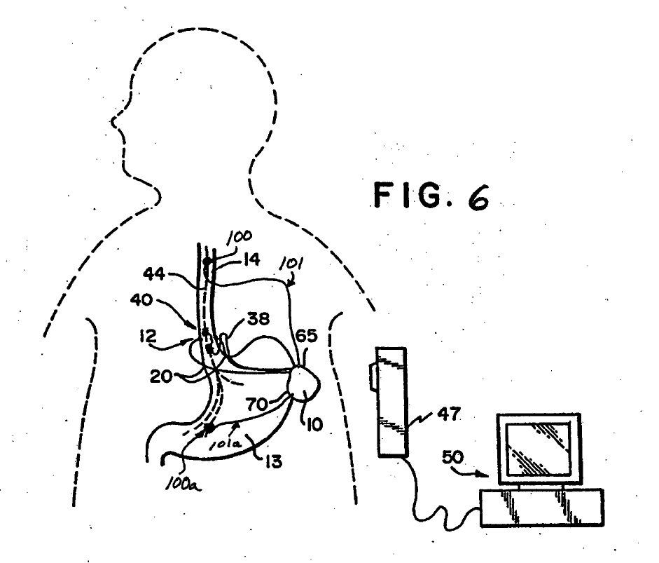

Having described the teachings of the '104 patent and the '480 patent, the improvement of the present invention will now be described with reference to FIG. 6. FIG. 6 shows an improvement of FIG. 2 by the addition of neural conduction blocks, as will be described. It will be appreciated that FIG. 3 disclosure can be similarly improved.

FIG. 6 shows an improved embodiment according to the present invention using a nerve conduction blocking electrodes 100, 100a positioned on the vagus nerve 44 (or its trunks). The blocking electrodes 100, 100a are positioned between the stimulating electrodes 40 of the prior art and organs to be shielded from the stimulation. For example, blocking electrode 100 is positioned between the heart and stimulating electrode 40. Blocking electrode 100a is positioned between-distal organs (intestinal organs, pancreas, gall bladder etc) and stimulating electrode 40. It will be appreciated that not both of proximal and distal electrodes 100, 100a need be placed or, if both placed, both functioning. The use of proximal and distal electrodes 100, 100a permits a physician to block proximally and distally, respectively, as needed to reduce adverse effects (e.g., vocal, cardiac or gastrointestinal) of organs impacted by the stimulation electrode 40.

Examples of electrode designs are shown in U.S. Pat. No. 4,979,511 to Terry, Jr. dated Dec. 25, 1990; U.S. Pat. No. 5,215,089 to Baker dated Jun. 1, 1993; U.S. Pat. No. 5,251,634 to Weinberg dated Oct. 12, 1993; U.S. Pat. No. 5,351,394 to Weinberg dated Oct. 4, 1994; U.S. Pat. No. 5,531,778 to Mashino-dated Jul. 2, 1996; and U.S. Pat. No. 6,600,956 to Mashino dated Jul. 19, 2003 (all incorporated herein by reference).

The blocking electrodes 100, 100a are connected by lead 101, 101a to a controller (e.g., the pulse generator 10 of FIG. 2) adapted, in a preferred embodiment, to generate, at electrodes 100, 100a, the blocking parameters that will be described hereafter. The blocking creates a neural block at the electrodes 100, 100a. With such blocking parameters at blocking electrode 100, impulses from the stimulating electrode are attenuated to avoid unintended interference with other organs.

A nerve block or neural conduction block is, functionally speaking, a reversible vagotomy. Namely, application of the block at least partially prevents nerve transmission across the site of the block. Removal of the block restores normal nerve activity at the site. A block is any localized imposition of conditions that at least partially diminish transmission of impulses.

The vagal block of electrodes 100, 100a is desirable since unblocked pacing may result in afferent vagal and antidromic efferent signals having undesired effect on organs innervated directly or indirectly by the vagus (e.g., undesirable cardiac response or vocal response). Further, the afferent signals of the stimulation electrode 40 can result in a central nervous system response that tends to offset the benefits of the stimulation electrode 40 thereby reducing effectiveness of vagal_stimulation.

The block may be intermittent and applied only when the vagus is stimulated by the stimulation electrode 40. The preferred nerve conduction block is an electronic block created by a signal at the vagus 44 (or its trunks) by an electrode 100, 100a controlled by the previously described control system. The nerve conduction block can be any reversible block. For example, cryogenics (either chemically or electronically induced) or drug blocks can be used. An electronic cryogenic block may be a Peltier solid-state device which cools in response to a current and may be electrically controlled to regulate cooling. Drug blocks may include a pump-controlled subcutaneous drug delivery.

With such an electrode conduction block, the block parameters (signal type and timing) can be altered by a controller and can be coordinated with the pacing signals to block only during pacing. A representative blocking signal is a 500 Hz signal with other parameters (e.g., timing and current) matched to be the same as the pacing signal). The precise signal to achieve blocking may vary from patient to patient and nerve site. The precise parameters can be individually tuned to achieve neural transmission blocking at the blocking site.

While an alternating current blocking signal is described, a direct current (e.g.,—70 mV DC) could be used. The foregoing specific examples of blocking signals are representative only. Other examples and ranges of blocking signals are described in the afore-mentioned literature (all incorporated herein by reference). As will be more fully described, the present invention gives a physician great latitude in selected stimulating and blocking parameters for individual patients.

Nerve conduction blocking permits longer stimulation pulse durations and intensities which would otherwise have adverse effects on other organs such as those of the cardiovascular or gastrointestinal systems.

As described, the parameters of the stimulating and blocking electrodes 40, 100 can be inputted via a controller and, thereby, modified by a physician. The blocking electrode can also be controlled by an implanted controller and feedback system. For example, physiologic parameters (e.g., heart rate, blood pressure, etc.) can be monitored. The blocking signal can be regulated by the controller to maintain measured parameters in a desired range. For example, blocking can be-increased to maintain heart rate within a desired rate range during stimulation pacing.

With the benefit of blocking as described, the stimulation therapy can be applied more regularly (e.g., intermittently throughout the day) and need not be limited to times when an onset of need for therapy (e.g., a sensed onset of an epileptic seizure) is detected. This eliminates the need for complicated and potentially unreliable event detection and permits the use of the therapy to avoid an event before it starts.

C. Teachings of Additional Prior Art

For ease of illustrating the present invention in a preferred embodiment for improving a prior art system for treating obesity and eating disorder in a second prior art embodiment, a recitation of the invention of U.S. Pat. No. 6,587,719 to Barret et al. dated Jul. 1, 2003 (the “'719 patent”) is first provided. The discussion of the '719 patent is made with reference to FIG. 7 which is the sole figure from the '719 patent (and there being no reference numbers for drawing elements in the '719 patent).

A generally suitable form of neurostimulator for use in the apparatus and method of the present invention is disclosed in. The specification of the '172 patent is incorporated herein in its entirety by reference.

According to the present invention, the patient is treated with bilateral stimulation of the right and left vagi branches at the supradiaphragmatic position of the vagus nerve, using neurostimulators (e.g., the NCP generator available from Cyberonics, Inc. of Houston, Tex. (Cyberonics)) placed, for example, via a left anterior thoracic incision. A standard Cyberonics Bipolar Lead nerve electrode, for example, is attached to the nerve generator after the patient's eating behavior is standardized and a stable dietary pattern is observed.

In dog tests conducted by the applicants of the '719 patent, the dietary pattern included twice-a-day feedings of approximately 400 grams of solid food with one scoop of soft meat product added to make the food more edible. During the surgical procedure, a threshold referred to herein as the retching threshold was documented while the animal was under anesthesia, based on the threshold value of the stimulus output current of the device at which the animal exhibited a retching or emetic response. The amount of current was adjusted to determine this threshold. Other parameters were left fixed at a frequency of 30 Hertz (Hz), a pulse width of 500 milliseconds (ms), and an on/off cycle of one minute on and 1.8 minutes off.

Following the implant of the bilateral nerve stimulators, the animals were allowed to stabilize. Once eating behavior returned to preoperative levels the vagal nerve stimulators were turned on in two canines. These two were given chronic intermittent bilateral nerve stimulation over a twenty-four hour period. Initial amplitude was set at approximately 1.0 to 1.5 milliamperes (mA) below the retching threshold, and adjusted thereafter. The retching thresholds in mA increased over a period of days.

According to the '719 patent, both chronic dogs behaved in the same manner. Initially there was no change in the eating behavior. Approximately seven to ten days later, while still being subjected to chronic intermittent bilateral nerve stimulation, eating behavior changed in both dogs. They demonstrated a lack of enthusiasm for their food, while maintaining normal behavior for all other aspects of laboratory life. Instead of consuming their meal in approximately five minutes, as had been their customary preoperative behavior, their meal consumption took between fifteen and thirty minutes. More striking was the observed manner in which they consumed the food; each of the two would eat a small portion, leave the food dish, walk around, and ultimately return to the food from what appeared to be more a case of instinct than desire.

The '719 patent states to make certain a real effect attributable to the bilateral stimulation was being observed, after a six week period in which the intermittent stimulation was maintained, and consistent, altered eating behavior of the dogs continued, the stimulation was turned off. A of remarkable change in eating behavior was observed in each dog in one week after stimulation was discontinued, each dog exhibiting a return to its normal eating pattern after a few to several days in which it enthusiastically consumed its entire meal. Then, both stimulators were turned back on to provide the chronic intermittent bilateral stimulation in each animal, and the eating pattern of the animal slowed once again after approximately 10 to 15 days to what had been observed in the postoperative period following such stimulation.

The '719 patent indicates further study was performed to determine whether unilateral stimulation would suffice, and whether a difference could be discerned between stimulation of the right vagus versus the left vagus. With only the left nerve stimulator turned for intermittent stimulation over a period of several days, no slowing in the animal's eating behavior was observed. The left stimulator was then turned off, and the latter testing was duplicated, this time using only right vagus nerve stimulation. Once again, after a period of several days of unilateral intermittent stimulation, no slowing of the animal's eating behavior was observed.

Finally, both nerve stimulator generators were turned back on and, after a period of several days of the bilateral stimulation, each of the animal's eating behavior reverted to the slowed pace that had been observed in the postoperative period following such stimulation. The applicants postulate that these tests demonstrate that bilateral chronic intermittent stimulation is effective to change eating behavior in animals, and this same treatment is expected to be effective in changing eating behavior in obese human patients and in human patients suffering from compulsive overeating disorder, whether or not the patient is obese in the more strict sense of that term.

Moreover, the '719 patent states the testing further demonstrated by use of acute as well as chronic stimulation that a positive response of satiety was the cause of the lack of interest of the animals in food, rather than a negative response of nausea or sick stomach. In the acute testing protocol the animals were not subjected to bilateral stimulation of the vagi until fifteen minutes to one half hour before feeding time, and throughout the meal. Such acute bilateral stimulation failed to change the eating behavior of the animals from normal baseline eating pattern to a demonstrably slowed eating pattern--change that would have been expected to occur if the stimulation had the effect of producing nausea. These tests tend to show that the slowed eating and apparent disinterest in food consumption is centrally mediated and the result of producing a sensation of satiety mimicking that which would occur after consumption of a full meal.

The characterization of the bilateral stimulation as being “intermittent” is made in the sense that the stimulation was performed following a prescribed duty cycle of application of the signal. The latter is a pulse signal, and is applied with a prescribed or preset or predetermined on-time of the pulses, followed by a prescribed or preset or predetermined off-time of the pulses, which could be the same as but in general is different from the on-time. It is possible, however, depending upon other parameters of the electrical pulse signal, that a continuous signal might be effective to produce the slowed eating behavior. It is also possible to use a single implanted nerve stimulator (pulse generator) with appropriate duty cycle to provide the bilateral stimulation of both vagal branches, right and left. Or the stimulation may be different for each branch and use different implanted stimulators. And although implanted stimulators are preferred, it is also possible to treat patients receiving clinical or in-hospital treatment by means of external devices that provide vagal stimulation via leads and electrodes implanted in the patient. Wholly implanted devices are preferred, however, because they allow patients to be completely ambulatory, and without interfering with routine daily activities.

In the '719 patent, two other dogs with bilateral stimulators were studied in a different fashion. Initially their stimulators were left off (inactive), and were only turned on just prior to challenging the animal with food, that is, a few minutes before the meal, and during the meal. No effect on eating behavior was observed in response to such acute bilateral vagus nerve stimulation. That is, each dog followed its normal or baseline preoperative eating behavior without noticeable or perceptible slowing.

Some differences from stimulator to stimulator in magnitude of current in the pulses of the electrical stimulation signal may be observed, and may be attributable to things such as patient impedance, variation of the vagus nerve from right to left or between patients, and variation in contact between the vagus and the electrode implanted thereon from implant to implant.

Although certain preferred embodiments and methods of treating and controlling eating disorders through vagal modulation according to the invention have been described herein, it will be apparent to those skilled in the field from a consideration of the foregoing description that variations and modifications of such embodiments, methods and techniques may be made without departing from the true spirit and scope of the invention. Accordingly, it is intended that the invention shall be limited only to the extent required by the appended claims and the rules and principles of applicable law.

D. Improvement of the Present Invention

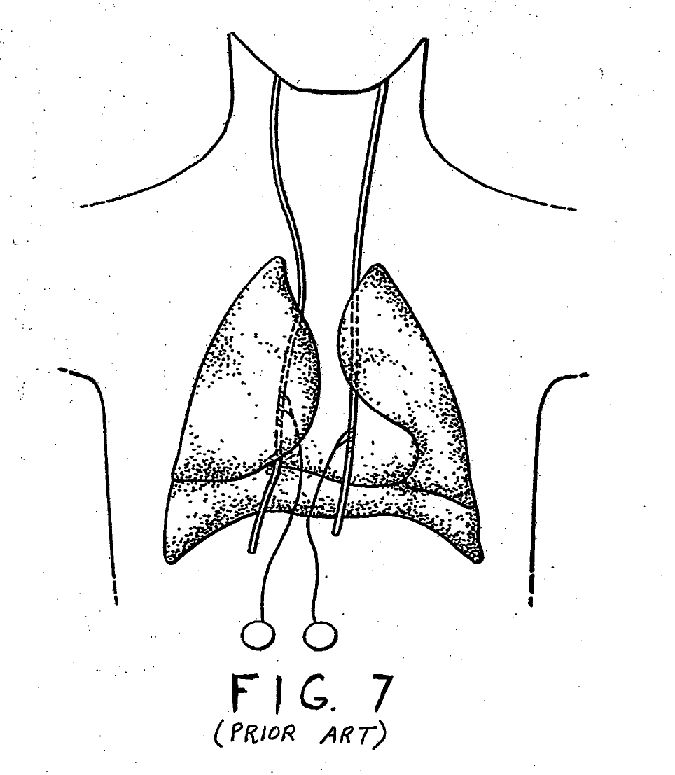

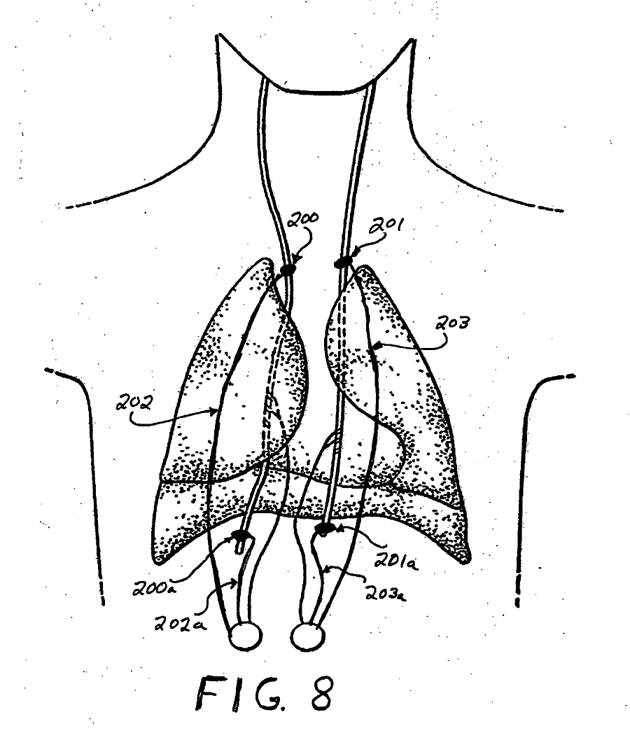

Having described the teachings of the '719 patent, the improvement of the present invention will now be described with reference to FIG. 8. FIG. 8 shows an improvement of FIG. 7 by the addition of neural conduction blocks.

The improvement includes the addition of neural conduction blocking electrodes 200, 201, 200a, 201a positioned on the nerves both proximally and distally to the stimulation electrodes of the '719 patent. The blocking electrodes 200, 201, 200a, 201a are connected by leads 202, 203, 202a, 203a to the same generators which provide signals to the stimulation electrodes. The generators are adapted to generate not only the stimulating signals of the '719 patent but also a neural blocking signal as described above and to direct the blocking signal along the leads 202, 203, 202a, 203a to the blocking electrodes 200, 201, 200a, 201a to block signal transmission on the nerve at the blocking sites.

As with the previously described embodiments, all of proximal and distal electrodes 200, 201, 200a, 201a need not be placed or, if all placed, all functioning. The use of proximal and distal electrodes 200, 201, 200a, 201a permits a physician to block proximally and distally, respectively, on either of the nerves as needed to reduce adverse effects (e.g., vocal, cardiac or gastrointestinal) of organs impacted by the stimulation electrode.

E. Teachings of Still Additional Prior Art

For ease of illustrating the present invention in a preferred embodiment for improving a prior art system for treating obesity and eating disorder in a third prior art embodiment, a recitation of the invention of U.S. Pat. No. 6,609,025 to Barret et al. dated Aug. 19, 2003 (the “'025 patent”) is first provided.

A generally suitable form of neurostimulator for use in the apparatus and method of the present invention is disclosed, for example, in U.S. Pat. No. 5,154,172 (the device also referred to from time to time herein as a NeuroCybernetic Prosthesis or NCP device (NCP is a trademark of Cyberonics, Inc. of Houston, Tex.)). Certain parameters of the electrical stimuli generated by the neurostimulator are programmable, preferably by means of an external programmer (not shown) in a conventional manner for implantable electrical medical devices.

Referring to FIGS. 9-13, the neurostimulator, identified in the drawing by reference number 110 is implanted in a patient 112, preferably in the abdominal region, for example, via a left laparotomy incision. For the preferred implementation and method of direct bilateral stimulation, lead-electrode pair 115, 116 is also implanted during the procedure, and the proximal end(s) of the lead(s) electrically connected to the neurostimulator. The lead-electrode may be of a standard bipolar lead nerve electrode type available from Cyberonics, Inc.

According to the preferred method of the '025 patent, the nerve electrodes 117, 118 are implanted on the right and left branches 119, 120, respectively, of the patient's vagus nerve at a sub-diaphragmatic location. The nerve electrodes are equipped with tethers for maintaining each electrode in place without undue stress on the coupling of the electrode onto the nerve itself. Preferably, the sub-diaphragmatic location of this coupling is approximately two to three inches below the patient's diaphragm 122 for each branch 119, 120.

Neurostimulator 110 generates electrical stimuli in the form of electrical impulses according to a programmed regimen for bilateral stimulation of the right and left branches of the vagus. During the implant procedure, the physician checks the current level of the pulsed signal to ascertain that the current is adjusted to a magnitude at least slightly below the retching threshold of the patient. Typically, if this level is programmed to a value less than approximately 6 mA, the patient does not experience retching attributable to VNS although variations may be observed from patient to patient. In any event, the maximum amplitude of the current should be adjusted accordingly until an absence of retching is observed, with a suitable safety margin. The retching threshold may change noticeably with time over a course of days after implantation, so the level should be checked especially in the first few days after implantation to determine whether any adjustment is necessary to maintain an effective regimen.

The bilateral stimulation regimen of the VNS preferably employs an intermittent pattern of a period in which a repeating series of pulses is generated for stimulating the nerve, followed by a period in which no pulses are generated. The on/off duty cycle of these alternating periods of stimulation and no stimulation preferably has a ratio in which the off time is approximately 1.8 times the length of the on time. Preferably also, the width of each pulse is set to a value not greater than about 500 .mu.s, and the pulse repetition frequency is programmed to be in a range of about 20 to 30 Hz. The electrical and timing parameters of the stimulating signal used for VNS as described herein for the preferred embodiment will be understood to be merely exemplary and not as constituting limitations on the scope of the invention.

The patient's eating behavior should be allowed to stabilize at approximately the preoperative level before the VNS regimen is actually administered. Treatment applied in the form of chronic intermittent bilateral nerve stimulation over each twenty-four hour period may be observed initially to result in no change in eating behavior of the patient. But after a period of several days of this VNS regimen, a discernible loss of interest in heavy consumption of food should occur. A typical result would be that mealtime consumption tends to stretch over a considerably longer period of time than that observed for the patient's preoperative behavior, with smaller quantities of food intake separated by longer intervals of no consumption in the course of a single meal. The VNS treatment should not affect normal behavior in other aspects of the patient's life. A complete suspension of the VNS regimen would result in a relatively rapid return to the previous overeating behavior, ending after resumption of the VNS regimen. Observations appear to indicate that treatment by bilateral stimulation may be safe and effective in changing eating patterns and behavior in obese human patients, and more generally in human patients suffering from compulsive overeating disorder.

According to the '025 patent, animal testing using bilateral VNS has tended to demonstrate that slowed eating and apparent lack of enthusiasm in food consumption is centrally mediated and the result of a positive response of inducing a sensation of satiety mimicking that which would occur after consumption of a full meal, rather than of a negative response of nausea or sick stomach.

The intermittent aspect of the bilateral stimulation resides in applying the stimuli according to a prescribed duty cycle. The pulse signal is programmed to have a predetermined on-time in which a train or series of electrical pulses of preset parameters is applied to the vagus branches, followed by a predetermined off-time. Nevertheless, continuous application of the electrical pulse signal may also be effective in treating compulsive overeating disorder.

Also, as shown in FIG. 10, dual implanted NCP devices 110a and 110b may be used as the pulse generators, one supplying the right vagus and the other the left vagus to provide the bilateral stimulation. At least slightly different stimulation for each branch may be effective as well. Use of implanted stimulators for performing the method of the invention is preferred, but treatment may conceivably be administered using external stimulation equipment on an outpatient basis, albeit only somewhat less confining than complete hospitalization. Implantation of one or more neurostimulators, of course, allows the patient to be completely ambulatory, so that normal daily routine activities including on the job performance is unaffected.

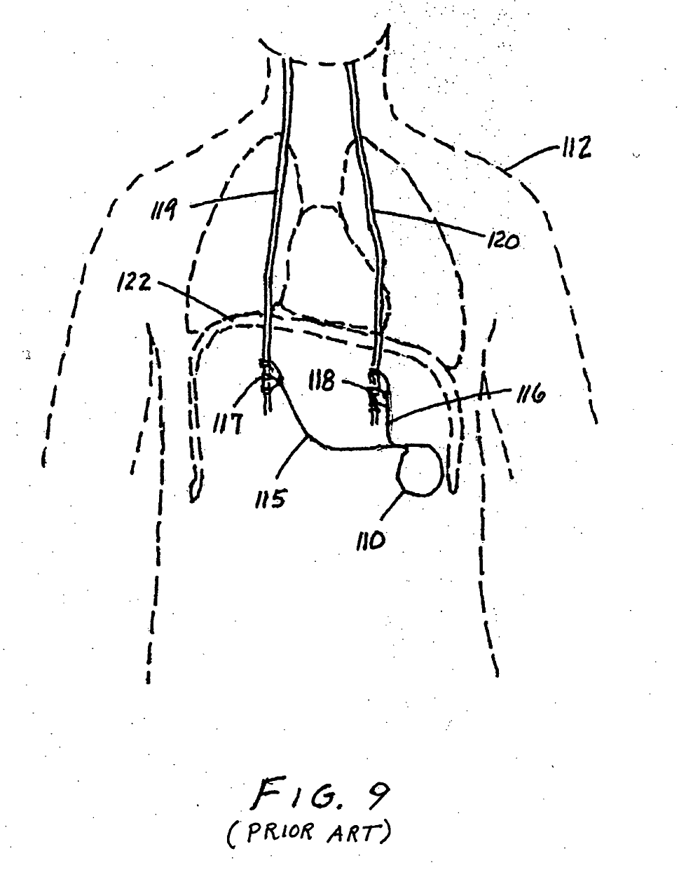

The desired stimulation of the patient's vagus nerve may also be achieved by performing unilateral sub-diaphragmatic stimulation of either the left branch or the right branch of the vagus nerve, as shown in FIG. 11. A single neurostimulator 110 is implanted together with a lead 115 and associated nerve electrode 117. The nerve electrode 117 is implanted on either the right branch 119 or the left branch 120 of the nerve, preferably in a location in a range of from about two to about three inches below the patient's diaphragm 122. The electrical signal stimuli are the same as described above.

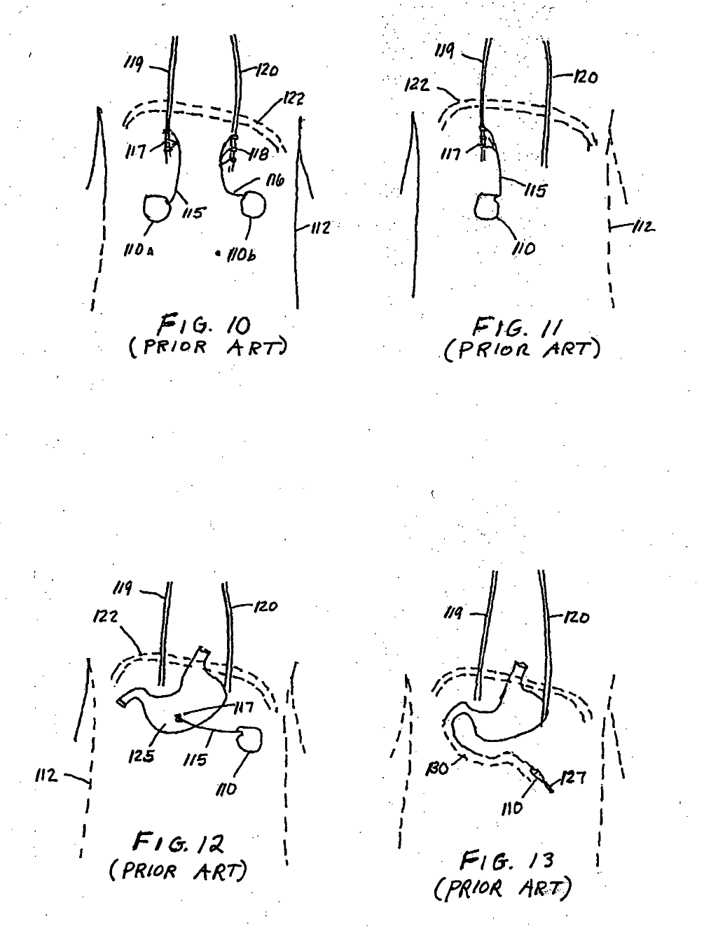

In a technique illustrated in FIG. 12, the signal stimuli are applied at a portion of the nervous system remote from the vagus nerve such as at or near the stomach wall 125, for indirect stimulation of the vagus nerve in the vicinity of the sub-diaphragmatic location. Here, at least one signal generator 110 is implanted together with one or more electrodes 117 subsequently operatively coupled to the generator via lead 115 for generating and applying the electrical signal internally to a portion of the patient's nervous system other than the vagus nerve, to provide indirect stimulation of the vagus nerve in the vicinity of the desired location. Alternatively, the electrical signal stimulus may be applied non-invasively to a portion of the patient's nervous system for indirect stimulation of the vagus nerve at a sub-diaphragmatic location.

In an arrangement shown in FIG. 13, the signal stimuli are applied remotely from electrical stimulating device 110 placed by an endoscope 127 from an area composing the GI tract 130.

F. Improvement of the Present Invention

Having described the teachings of the '025 patent, the improvement of the present invention will now be described with reference to FIGS. 14-18. These figures show an improvement of FIGS. 9-13 by the addition of neural conduction blocks as previously described.

In FIG. 14, the improvement to the embodiment of FIG. 9 includes the addition of proximal neural conduction blocking electrodes 300, 300 a positioned on the nerves proximally to the stimulation electrodes 117, 118. Distal neural conduction blocking electrodes 301, 301a are positioned on the nerve distally to the stimulation electrodes 117, 118. The blocking electrodes 300, 300a, 301, 301a are connected by leads 302, 302a, 303, 303a to the same generator 110 provides signals to the stimulation electrodes 117, 118. The generator 110 is adapted to generate not only the stimulating signals of the '025 patent but also a neural blocking signal as described above and to direct the blocking signal along the leads 302, 302a, 303, 303a to the blocking electrodes 300, 300a, 301, 301a to block signal transmission on the nerve at the blocking sites.

As with the previously described embodiments, all of proximal and distal electrodes 200, 201, 200a, 201a need not be placed or, if all placed, all functioning. The use of proximal and distal electrodes 200, 201, 200a, 201a permits a physician to block proximally and distally, respectively, on either of the nerves as needed to reduce adverse effects (e.g., vocal, cardiac or gastrointestinal) of organs impacted by the stimulation electrode.

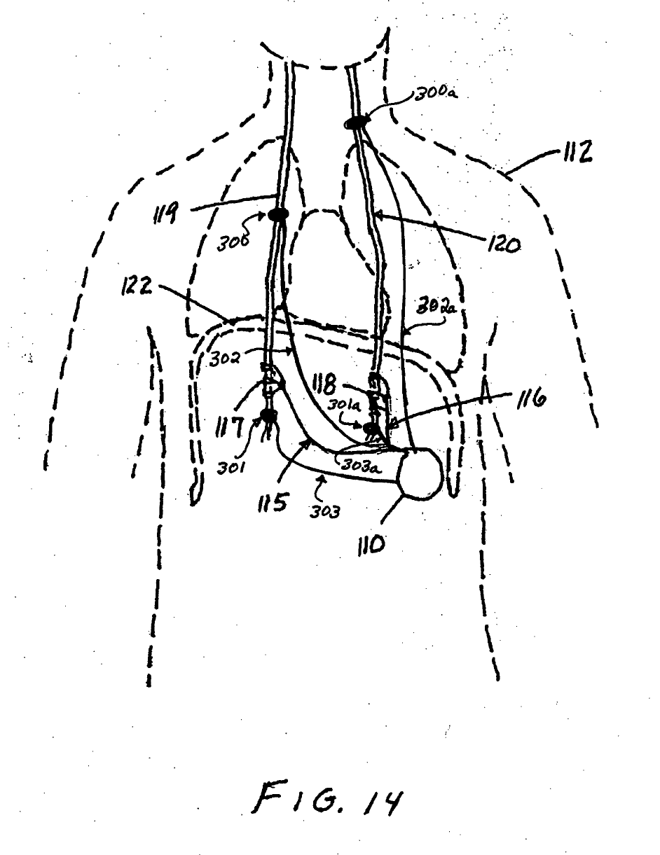

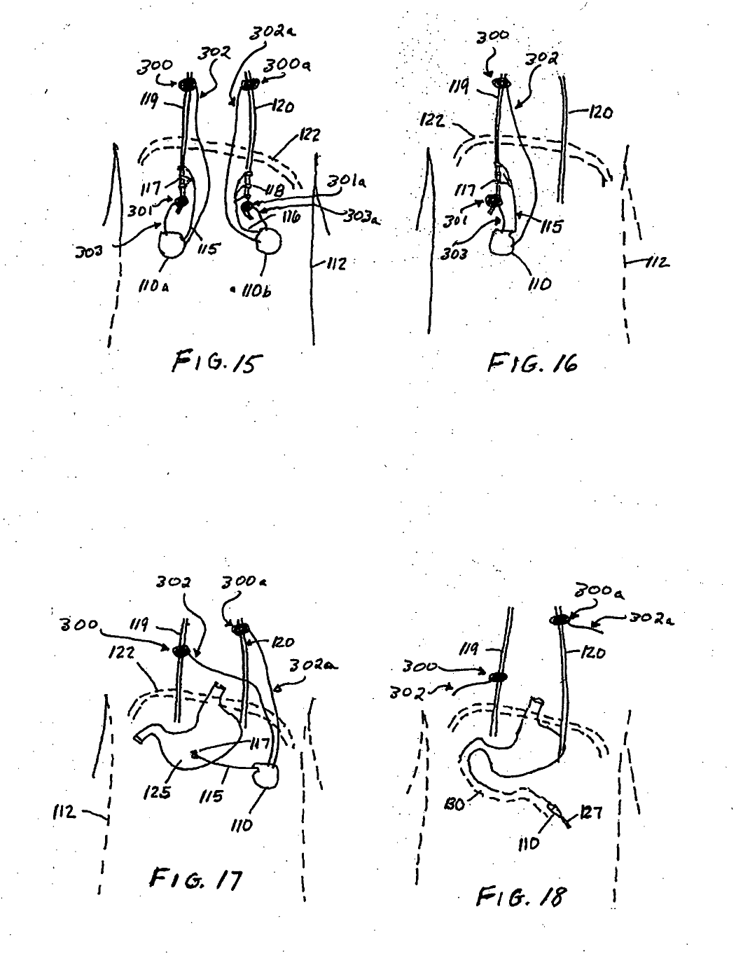

FIG. 15 improves over FIG. 10 in the same manner as FIG. 14 except leads 302, 303 from the right (from the patient's perspective) right blocking electrodes 300, 303 are directed to a right generator and leads 302a, 303a from the left blocking electrodes 300a, 301a are directed to the left generator 110b. In FIG. 16, there are only right blocking electrodes 300, 303 since there is only a right stimulation electrode 117. In FIG. 17, only blocking electrodes 300, 300a are shown. It will be appreciated blocking electrodes could be placed on nerve trunks distal to the site of the stimulation electrode 117. Similarly, in FIG. 18, only blocking electrodes 300, 300a are shown. Leads 302, 302a can be connected to internal or external generators (not shown).

With the foregoing detailed description of the present invention, it has been shown how the objects of the invention have been attained in a preferred manner. Modifications and equivalents of disclosed concepts such as those which might readily occur to one skilled in the art, are intended to be included in the scope of the claims which are appended hereto.

1. A method of treating patients with eating disorders by use of a therapy which alleviates a symptom of the disorder, which comprises the steps of:
responding to a specific eating disorder of interest by applying a predetermined stimulating signal to the patient's vagus nerve appropriate to alleviate said symptom of the eating disorder of interest; and
applying a neural conduction block to the vagus nerve at a blocking site with said neural conduction block selected to at least partially block nerve impulses on said vagus nerve at said blocking site.

2. A method according to claim 1 wherein said neural conduction block is applied to said nerve between a location of application of said stimulating signal and an organ to be shielded from adverse effects of said stimulating signal.

3. A method according to claim 1 wherein said neural conduction block is applied during application of said stimulating signal.

4. A method according to claim 1 wherein application of said neural conduction block is variable by a controller to alter a characteristic of said block.

5. A method according to claim 1 wherein said neural conduction block is a cryogenic block.

6. A method according to claim 1 wherein said neural conduction block is a pharmocologic block.

7. A method according to claim 1 wherein said neural conduction block is an electrical conductive block.

8. A method according to claim 1 comprising detecting a preselected event indicative of an imminent need for treatment of the specific eating disorder of interest and said responding includes responding to the detected occurrence.

9. A method according to claim 1, wherein the disorder is compulsive eating to excess, and said stimulating signal is predetermined to produce a sensation of satiety in the patient.

10. A method according to 1, wherein said stimulating signal is applied to the patient's vagus nerve by a nerve electrode implanted on the nerve in the vicinity of the patient's stomach.

11. Apparatus for treating patients with eating disorders by application of a modulating signal to the patient's vagus nerve to stimulate or inhibit neural impulses and produce excitatory or inhibitory neurotransmitter release by the nerve according to the specific nature of the eating disorder, comprising:
a neurostimulator for generating an electrical output signal in response to activation thereof, said neurostimulator means including:
a stimulating electrode responsive to generation of the electrical output signal by said neurostimulator means for application thereof as said modulating signal to the patient's vagus nerve.
an electrically controllable neural conduction electrode adapted to be electrically coupled to said vagus nerve at a blocking site between said stimulating electrode and an organ to be shielded from adverse effects of said stimulating electrode; and
a blocking signal generator for generating a blocking signal selected to at least partially block nerve impulses on said vagus nerve at said blocking site.

12. An apparatus according to claim 11, a sensor for detecting the occurrence of a selected event indicative of the need for imminent treatment of the eating disorder, and including an activator responsive to the detection for activating said neurostimulator.

13. An apparatus according to claim 11 wherein said neural conduction block is applied during application of said stimulating signal.

14. A method according to claim 11 wherein application of said neural conduction block is variable by a controller to alter a characteristic of said block.

15. A method according to claim 11 wherein said neural conduction block is a cryogenic block.

16. A method according to claim 11 wherein said neural conduction block is a pharmocologic block.

17. A method according to claim 1 1 wherein said neural conduction block is an electrical conductive block.

18. A neurostimulator for treatment of eating disorders in human patients, comprising an implantable electrical signal generator,
programming means for selectively programming the parameters of the electrical signal generated by the generator to modulate electrical activity of the patient's vagus nerve to evoke different sensations by the patient according to the nature of the eating disorder being treated,
means for applying the generated signal to the patient's vagus nerve, means for detecting a symptom of the eating disorder being treated to activate the generation of the programmed electrical signal by the generator;
an electrically controllable neural conduction electrode adapted to be electrically coupled to said vagus nerve at a blocking site between said stimulating electrode and an organ to be shielded from adverse effects of said stimulating electrode; and
a blocking signal generator for generating a blocking signal selected to at least partially block nerve impulses on said vagus nerve at said blocking site.

19. A method for treating and controlling compulsive eating disorders characterized by compulsive action exhibited by the individual suffering from the disorder, which comprises the steps of:
detecting a known symptom of the eating disorder of interest indicative of imminent need for intervention to control the disorder,
responding to the detected occurrence of said symptom by modulating the vagal activity of the individual suffering the eating disorder to treat the symptom and deter the compulsive action associated with the disorder;
applying a neural conduction block to the vagus nerve at a blocking site with said neural conduction block selected to at least partially block nerve impulses on said vagus nerve at said blocking site.

20. A method of treating patients with eating disorders, which comprises the steps of:
detecting the commencement of a customary mealtime according to the patient's circadian cycle, as being indicative of an imminent need for treatment of the patient's eating disorder;
responding to the detected commencement of the customary mealtime by applying a predetermined stimulating signal to the patient's vagus nerve appropriate to alleviate the patient's eating disorder; and
applying a neural conduction block to the vagus nerve at a blocking site with said neural conduction block selected to at least partially block nerve impulses on said vagus nerve at said blocking site.

21. A method of treating patients with an eating disorder associated with compulsive refusal to eat, which comprises the steps of:
detecting the commencement of a preselected event indicative of an imminent need for treatment of the patient's eating disorder;
responding to the detected occurrence of the preselected event by applying a predetermined stimulating signal to the patient's vagus nerve appropriate to alleviate the patient's eating disorder, said stimulating signal being predetermined to suppress a sensation of satiety in the patient; and
applying a neural conduction block to the vagus nerve at a blocking site with said neural conduction block selected to at least partially block nerve impulses on said vagus nerve at said blocking site.

22. Apparatus for the treatment of patients suffering from eating disorders, in which the apparatus has an implantable neurostimulator device adapted to generate a preprogrammed electrical output signal upon activation of the device, and an implantable electrical lead assembly connectable to the neurostimulator device and with an electrode adapted to be secured to the patient's vagus nerve for electrical excitation of the nerve to modulate its electrical activity, characterized in that:
the apparatus further includes sensor means electrically coupled to the neurostimulator device for detecting the occurrence of a predetermined event associated with the eating disorder of interest;
activator means electrically coupled to the neurostimulator device and responsive to detection of the predetermined event to activate the neurostimulator device to apply its preprogrammed electrical output signal to the electrical lead assembly for electrical excitation of the vagus nerve to modulate the electrical activity of the nerve so as to stimulate or inhibit neural impulses and produce excitatory or inhibitory neurotransmitter release by the vagus nerve according to the specific nature of the eating disorder; and
an electrically controllable neural conduction electrode adapted to be electrically coupled to said vagus nerve at a blocking site between said stimulating electrode and an organ to be shielded from adverse effects of said stimulating electrode; and
a blocking signal generator for generating a blocking signal selected to at least partially block nerve impulses on said vagus nerve at said blocking site.

23. A new use for a neurostimulator device adapted to be implanted in a human patient, in which the device comprises an electrical signal generator which is programmable to generate an electrical output signal having selected signal parameters, and an electrical lead adapted to be connected to the signal generator including an electrode adapted to be implanted on the patient's vagus nerve to modulate the electrical activity of the nerve in response to application of the programmed electrical output signal from the signal generator to the lead, the new use of the neurostimulator device characterized by therapeutic treatment and control of an eating disorder of the patient, including the steps of:
detecting the occurrence of a predetermined event associated with the eating disorder of interest;
upon detection of the predetermined event, activating the neurostimulator device to apply the programmed electrical output signal of the signal generator to the lead for electrical excitation of the vagus nerve to modulate the electrical activity of the nerve so as to stimulate or inhibit neural impulses and produce excitatory or inhibitory neurotransmitter release by the vagus nerve according to the specific nature of the eating disorder; and
applying a neural conduction block to the vagus nerve at a blocking site with said neural conduction block selected to at least partially block nerve impulses on said vagus nerve at said blocking site.

24. A method of controlling the function of a neurostimulator device adapted to be implanted in a human patient, including the following steps:
selecting parameters including pulse amplitude, pulse width and on and off times of an electrical output signal of a pulse generator to develop an electrical signal for treating an eating disorder of the patient for application to a lead/electrode assembly implanted on the vagus nerve of the patient to appropriately modulate the electrical activity of the nerve;
programming the pulse generator after implantation to set the selected parameters of its electrical output signal for treatment of said eating disorder; and
applying a neural conduction block to the vagus nerve at a blocking site with said neural conduction block selected to at least partially block nerve impulses on said vagus nerve at said blocking site.

25. A method of treating patients for obesity, which comprises the steps of:
performing bilateral stimulation of the patient's vagus nerve by applying a stimulating electrical signal directly and intermittently to the right and left vagi, wherein the parameters of said signal are predetermined to produce a sensation of satiety in the patient; and
applying a neural conduction block to the vagus nerve at a blocking site with said neural conduction block selected to at least partially block nerve impulses on said vagus nerve at said blocking site.

26. A method of treating patients for obesity, which comprises the steps of:
bilaterally stimulating the patient's vagus nerve by chronically applying a stimulating electrical signal intermittently to the right and left vagi, the parameters or said signal being selected to produce a sensation of satiety in the patient; and
applying a neural conduction block to the vagus nerve at a blocking site with said neural conduction block selected to at least partially block nerve impulses on said vagus nerve at said blocking site.

27. A method of treating patients for obesity, which comprises the steps of:
implanting separate nerve stimulator generators into the patient; bilaterally stimulating The patient's vagus nerve by applying a stimulating electrical signal intermittently to the right and left vagi from said implanted separate nerve stimulator generators, the parameters of said signal being selected to produce a sensation of satiety in the patient; and
applying a neural conduction block to the vagus nerve at a blocking site with said neural conduction block selected to at least partially block nerve impulses on said vagus nerve at said blocking site.

28. A method of treating patients for obesity, which comprises the steps of:
implanting nerve stimulator generator apparatus into the patient;
bilaterally stimulating the patient's vagus nerve by applying a stimulating electrical signal intermittently to the right and left vagi from said implanted nerve stimulator generator apparatus, the parameters of said signal being selected to produce a sensation of satiety in the patient; and
applying a neural conduction block to the vagus nerve at a blocking site with said neural conduction block selected to at least partially block nerve impulses on said vagus nerve at said blocking site.

29. A method of treating patients for obesity, which comprises the steps of:
implanting separate nerve stimulator generators into the patient;
bilaterally stimulating the patient's vagus nerve by applying a stimulating electrical signal in the form of a pulse signal having a prescribed on-off duty cycle continuously to the right and left vagi from said implanted separate nerve stimulator generators, so that pulses are applied during the on portion of said duty cycle and not during the off portion of said duty cycle, the parameters of said signal being selected to produce a sensation of satiety in the patient; and
applying a neural conduction block to the vagus nerve at a blocking site with said neural conduction block selected to at least partially block nerve impulses on said vagus nerve at said blocking site.

30. A method of treating patients for compulsive overeating, which comprises the steps of:
directly stimulating the left and right branches of the patient's vagus nerve simultaneously with electrical pulses in a predetermined sequence of a first period in which pulses are applied continuously, alternating with a second period in which no pulses are applied; and
applying a neural conduction block to the vagus nerve at a blocking site with said neural conduction block selected to at least partially block nerve impulses on said vagus nerve at said blocking site.

31. A method of treating obese patients by applying a therapy to reduce the patient's appetite, which comprises the steps of:
bilaterally stimulating the left and right branches of the patient's vagus nerve with an electrical signal applied directly or indirectly to both of said branches at a location below the patient's diaphragm, including programming electrical and timing parameters of said electrical signal, to give the patient a sensation of satiety and thereby induce weight loss of the patient; and
applying a neural conduction block to the vagus nerve at a blocking site with said neural conduction block selected to at least partially block nerve impulses on said vagus nerve at said blocking site.
